(19) United States
(12) Patent Application Publication
(10) Pub. No.: US 2005/0019750 A1
(43) Pub. Date: Jan. 27, 2005

(54) Method and device for isolating rna samples

(76) Inventor: Gottfried Brem, Hilgertshausen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/491,702

(22) PCT Filed: Oct. 7, 2002
(86) PCT No.: PCT/EP02/11223

(30) Foreign Application Priority Data
Oct. 18, 2001 (EP) 01 123 988.6

(51) Int. Cl. C12Q 1/70; C07H 21/04; C12M 1/34

(52) U.S. Cl. 435/5; 435/287.2; 536/23.72

(57) ABSTRACT

The invention concerns a device for isolating RNA samples from animals. This device has a sample collecting container and sampling means with means for protection against RNA-destroying enzymes. The invention concerns in particular a method for isolating RNA in biological samples which enables the search for RNA viruses in new-born babies.

The present invention relates to a device for isolating RNA samples from animals, including a sample collecting container and a sample extraction means, whereby both in the sample collecting container and also in/on the sample extraction means, agents are provided to protect against RNA-degrading enzymes. The present invention relates in particular to a process for isolating RNA from biological samples, and for screening animal herds/populations for pathogens.

Newborn livestock currently have manually written or pre-printed identifications fitted to confirm or to prove their identity and origin (plastic bands, ear tags, rings etc.), to prevent confusion with other individuals born in the same locality and so that the neonates can be reliably attributed to their mothers. One problem with these externally applied identifications is however that they can be lost, exchanged either accidentally or even intentionally or it can no longer be possible to allocate them correctly because documentation is missing. In such instances, the reliable reconstruction of the identity of the neonate and an attribution to a mother is in most cases no longer possible.

To resolve this problem, a device is proposed in PCT/EP98/03075, with which a biological sample containing DNA is taken at the same time as the ear tag is put in place, said DNA sample being subsequently possibly used for example to establish the individually typical genetic fingerprint or an SNP signature of the individual.

In the context of the birth and the marking of animals prescribed in almost every country, samples can easily be obtained and stored with this device and then used to test the genetic situation of neonates.

With regard to the epidemic diseases currently occurring in livestock, there is however an increased need to test animal herds or entire animal populations for diseases generally. A hitherto unresolved problem in this area however consists of obtaining from the neonates in a herd or even a population, samples of adequate quality and above all reliability with regard to the attribution to the individuals, so that even pathogens which are extremely difficult to detect for reasons of instability, can be covered.

Comprehensive neonate screening would enable a large number of diseases to be detected at an early stage and thus also treated or controlled in a specific manner, or preventive measures to be introduced.

One task of the present invention thus consists of overcoming the known disadvantages of the state of the art and providing a possibility for capturing pathogens which are difficult to detect because of their instability.

This task is resolved by a device which contains a sample collecting container and a sample extraction means whereby there are agents to protect against RNA-degrading enzymes both in the sample collecting container and on or in the sample extraction means.

In the studies which in the end led to the present invention, it was then found that it is clearly not sufficient, where the isolation of RNA from an individual is wanted by employing conventionally used devices, to provide only one agent for protecting against RNA-degrading enzymes in the containers provided for holding the sample. Instead, immediately upon contact of the sample extraction means with the biological sample it must be ensured that RNA-degrading enzymes cannot develop any activity so that the samples containing RNA are still suitable for tests even after prolonged storage. In accordance with the invention, therefore, the sample extraction means also contains a substance of this kind so that even during the short time for which the sample extraction means is introduced in the sample container when extracting the sample, it is ensured that no substantial degradation of RNA occurs. A procedure of this kind also ensures that, even during prolonged storage, the RNA remains stable in the container and is not subject to any degradation.

The sample collecting container and the sample extraction means can de facto be in any form with which a biological sample can be extracted and stored.

In accordance with a preferred embodiment, the sample container and the sample extraction means are in the form described in PCT/EP98/03075, which is incorporated herein by reference for the further clarification of the device called Typi-Fix® ear tags for extracting biological samples. Typi-Fix® ear tags can for example be filled or coated with any known RNAse inhibitor, such as trizol or RNAlater™ for example (AMS Biotechnologie GmbH, Wiesbaden, Germany), which are introduced into the sample collecting container and onto or into the sample extraction means. To ensure that the fluid in the sample collecting container is always on the base of the collecting container and thereby reliably comes into contact with the subsequently extracted tissue sample, the agent to protect against RNA-degrading enzymes can be held on the base for example by a membrane fitted in the container near the base or a carrier for the agent can be provided in the container, for example a porous carrier, such as a sponge of a shape modified to that of the sample collecting container.

To provide the sample extraction means with the agent to protect against RNA-degrading enzymes, it can for example be coated with the appropriate agent. Alternatively, if for example the sample extraction means is in the form of a hollow point, the point can be filled with the agent and sealed with an appropriate means, such as a membrane. This can for example be achieved by the ultrasound welding of a covering membrane with the conical head of the spike or in any other appropriate way by bonding etc. Ear tags prepared in this way can be stored for years without any deterioration of the preservative's function.

The advantage of filling both the hollow point of the spike of the male ear tag and the sample collecting container holding this point lies in the fact that the tissue sample thereby comes into direct contact with the preservative fluid from both sides at the time of stamping and is thereby not only wetted with liquid but quickly soaked and drenched by the liquid. With this process, the RNAses are immediately inhibited and the RNA in the tissue samples protected and maintained to the extent that even after prolonged storage the detection of certain transcripts in the RNA pool is still possible.

In accordance with one embodiment of the invention, a process for extracting biological samples containing RNA is provided in which the previously described device is used. The process is suitable in particular for screening animal herds or entire animal populations for pathogens, such as RNA viruses.

With the help of the process developed here and the tissue-sampling ear tags modified for RNA extraction, it is then possible, for example, to carry out eradication programmes for viral diseases where neonates already carry the RNA virus. By analysing the tissue/RNA samples extracted, all virus carriers can be identified and removed from the herd. In this way, the infection chain would be interrupted and the infection eradicated from the stock.

In accordance with a preferred embodiment, the present invention provides a process for detecting BVD in animals. For this, the sample containing the RNA virus must be extracted within the first few days of life, in particular before any antibodies absorbed with the colostrum take effect. Antibodies in colostrum can adversely affect virus detection from about the 3rd day of life through to the end of the 3rd month of life in such a way that a negative finding in this period is not meaningful and there is hence a “diagnostic gap”. The present invention therefore for the first time enables neonates to be tested for this disease so that the “gap” existing to date can be closed.

In accordance with the Livestock Circulation Order and EU Directive, all calves born in the EU must be identified with two ear tags within the first week of life. If this identification is actually carried out soon after the birth and ear tags are used which preserve the RNA in the sample stamped out of the ear, material for testing can be obtained from all calves in a population, with virus diagnosis possible from such material.

Alternatively and in addition, blood samples can also be extracted without any problem from the umbilical cord, tissue samples from the umbilical cord and parts of the placenta, mucous membrane swab samples etc. by people present at the birth using sample collecting containers from the birth sampling kit. The decisive advantage here is that all these sample collecting containers and thus all samples extracted can be obtained without any uncertainty and without any mix-up by the uniform and identical identification of the animal by means of ear tags and the container thanks to an identification printed on it, and in the laboratory at a minimal cost the identity can be further processed without error (for example, identification with bar code reader).

In this way, it is possible also to discover all persistently infected animals who could spread the infection throughout the herds once again. If these animals and their mothers are removed, no further re-infection occurs and after 1 to 3 years the individual herds and finally then also the relevant virus carriers should be eliminated and eradicated from the entire population. From an economic viewpoint, this would be very important for livestock breeding. According to information from the Bayersiche Landestierdrztekammer [Bavarian Regional Chamber of Veterinary Surgeons], BVD/MD in Bavaria is the bovine infectious disease which causes the greatest economic losses. There are model calculations which set the damage caused by BVD/MD at DM 50 to 100 per birth. This means, for example, for the Bavarian beef cattle population with an estimated 1.5 million births per year, damage of around DM 100 million. In addition to the economic aspects, the eradication of BVD/MD infections from our beef cattle would be an extremely desirable situation also in terms of animal welfare as the otherwise afflicted animals and those dying from the disease could be spared a great deal of pain, suffering and harm.

Other viruses which can be transmitted to neonates via the placenta or uterus or by any other route before, during or immediately after birth, can be tested for using the samples extracted and preserved in the sample collecting containers. This applies in particular to other RNA viruses too, for example PRRS (porcine reproductive and respiratory syndrome), TGE (transmissible gastro-enteritis) etc.

With regard to the individuals to be tested, all mammals can be considered, and for certain devices and tests humans too, and because of the economic interest in particular livestock, such as ungulates generally, for example beef cattle, sheep, pigs, horses etc., are of particular interest.

To carry out the process, in particular a set of sampling devices can be provided for various samples which are easy to obtain at the time of birth. These samples can be kept and furthermore also appropriately preserved for processing at a later date or for long-term storage. To avoid mistakes or mix-ups, the individual parts of the sampling set are connected to each other and packed together until immediately before use. They are separated from each other only when used (at any intended break points). All parts are pre-identified unmistakably and permanently (for example by laser labelling) and all the parts bear both the same analogue number for visual identification and also an associated barcode, 2-D code or other machine-readable identification (for example Find the Dot™).

The number of the sampling set is allocated to the mother's identity number and also linked to it genetically via the mother's sample.

A sampling set comprises labelled device and sample collecting container and identification components all identified and in accordance with the invention.

Sample collecting containers:
Sample collecting container with preservative for tissue samples for isolating DNA;
Sample collecting container with special preservative (for example trizol, RNAlater etc.) for tissue samples for isolating RNA;
Sample collecting container with cell culture solution containing antibiotics and antimycotics for storing vital cells from tissue samples from animals for cell analysis (for example viruses) and cloning;
Sample collecting container for blood stem cells from the umbilical cord with suitable stabilisers for preparation for the deep-freeze storage of such cells as a source of stem cells;
Sample collecting container for blood swabbed from the umbilical cord;
Sample collecting container for samples obtained from parts of the maternal or foetal placenta.
Sample collecting container for parts of the umbilical cord cut at birth;
ample collecting container with preservative for blood obtained by puncture of the blood vessels;
Sample collecting container for swab samples extracted from mucous membrane or skin smears or secretions (nose, mouth, eye, ear, genitalia etc.);
Sample collecting container for faeces (meconium) or urine samples obtained from the neonate;
Sample collecting container for hairs with hair roots obtained by combing or plucking;
Sample collecting container for suitable reference samples containing DNA, obtained from the mother (saliva, mucous membrane, blood, hair, tissue sample etc.); and alternatively if possible/wanted
Sample collecting container for a sample (containing DNA) from the father (insofar as he is available or accessible) of the neonate for checking descent;
Sample collecting container for a sample containing DNA from the genetic parents of the neonate for checking descent in embryo/gamete transfer programmes.

Identification components:
Adhesive labels with the identity number which can be pulled off and can be used for the labelling of transport packaging, documents, other collection vessels etc. or of the individual;
Documentation sheets with identity number in which all relevant data of the birth is recorded and thus is allocated unambiguously to the neonate; and
Plastic bands which bear the identification and are designed so that they cannot be re-opened once they have been closed, without destroying them, or
Metal tags, rings and such like which can be inserted in the skin; or
Ear tags which can be inserted in one or both ears; or
Other identification systems such as electronic chips (RFID), boluses, transponders or other devices which can bear the identity number; or
Reference component with the identification number of the neonate as identification which can be left for the mother or the owner of the animal.

For special applications, the set can be expanded or reduced as appropriate. Any user can define which sample collecting containers and identification systems are to be included in the set. The unique identification number of the neonate can be linked electronically with any existing data or patient documentation systems and stored there.

The sample collecting containers are designed such that the samples can be extracted simply and by people without medical training. The only exception is the—optional—taking of blood samples by the puncturing of blood vessels. This in turn is only necessary if serum parameters or other blood values are to be established. This procedure however falls within the scope of the veterinary/medical care of neonates and is supported by the sampling set in that appropriately pre-identified sample collecting containers are provided.

From all the preserved samples, DNA can also be isolated during the processing or testing if there should be any doubts or on a routine basis, and thus the identity of the origin of the sample confirmed and checked. It can thus be ensured that, during testing, the individual samples are actually attributed to the correct neonates.

Furthermore, by comparing the DNA of the neonate and mother, it can be ensured that the neonate does actually come from the tested mother and has not been confused with another. Even in situations such as those resulting from embryo transfer, where the genetic mother is thus not the mother giving birth to the neonate, a definite link can be produced and established by the DNA analysis of placental maternal and foetal tissue.

If there is a sample containing DNA from the father or if one can be obtained, the definitive confirmation of both parents is possible very simply and inexpensively.

The present invention can equally be used for obtaining vital cells from an individual. To do so, a cell culture medium is prepared in the sample collecting container and in the sample extraction means, preferably the hollow point of the spike plate, preferably in the same way as the agent for inhibiting RNAses, so that when the sample is extracted, the biological material immediately comes into contact with a medium so that its vitality can be maintained.

The medium contains preferably antibiotics and/or antimycotics to prevent contamination of the culture. The medium can likewise already contain agents which enable the cells to be frozen while maintaining their viability, such as for example glycerol or DMSO.

In this way, it is not only possible to keep cells vital and store them, and to use these cells as necessary after prolonged storage for further experiments too, such as cloning. Additionally, the structure of all existing antigens, be they of pathogens or endogenous antigens, is maintained and can be proven if wanted with the appropriate means.

The tissue samples stored in the sample collecting containers can be used in a culture at any time and tested as desired, for example in virus diagnosis using FACS analysis. The tissue sample can be treated to improve processing using collagenase or other enzymes, to break down the tissue structure and to thin out the cells.

### EXAMPLE 1

Extraction and Testing of RNA from Skin Samples or Other Samples Containing RNA Obtained with the Sample Collection set.

Eradication of BVD/MD (Bovine Virus Diarrhoea/Mucosal Disease).

Sample of tissue and secretions were taken from neonate calves within the first days of life using trizol and RNAlater Typi-Fix® ear tags. The filled sample collecting containers with the tissue sample introduced into the preservative were stored for up to 6 weeks before analysis.

The tissue samples were removed from the sample collecting containers for RNA extraction.

1. Processing Using Classic Methods

RNA Isolation

Addition of 100 μl chloroform, >15 s vortex, 2-3 min, RT.

Centrifuge for 15 min, 10,000 g, 4° C.

Remove aqueous phase on top and incorporate in 0.25 ml isopropanol.

Centrifuge off for 10 min at RT, then for 10 min at 10,000 g, 4° C.

Pour off excess, wash with 0.5 ml 75% ethanol.

Centrifuge for 5 min at 7,500 g.

Pour off excess and dry open on ice.

Reverse transcription (RT) and polymerase chain reaction (PCR)

The BVDV RNA isolated from the ear stamps in RNAlater™ with peqGOLD Trifast was converted into complementary DNA (cDNA) by means of reverse transcriptase and amplified in the same preparation in a polymerase chain reaction (one tube RT PCR) (Pfeffer et al. Vet Res Commun. 24 (7) (2000 November), 491-503; Kuhne S, Diss. med. vet. Munich).

1 to 5 μl RNA (1-3 μg total RNA) and 20 to 100 μmol each of forward and reverse primer (primers 324 and 326, Vilczek et al., Arch. Virol. 136: 309-323) was transferred into a 0.2 ml reaction vessel and made up to a volume of 20 μl with water treated with diethylpyrocarbonate (DEPC). To this preparation I, 80 μl of preparation II was pipetted, made of the following components:
0.5 μl reverse transcriptase (RAV-2, 20 U/μl; Amersham Pharmacia Biotech);
0.5 μl Taq polymerase (AmpliTaq DNA polymerase, 5 U/μl; Perkin-Elmer Biosystems);
0.5 μl Taq extender PCR additive (5 U/μl; Strategene);
2.0 μl nucleotide mix (dNTP mix: 10 μmol/ml each of dATP, dCTP, dGTP, dTTP; MBI Fermentas);
0.5 μl dithiothreitol (DTT, 100 mM; Roche);
0.5 μl RNAse inhibitor (RNAsin, 40 U/μl; Promega);
10.0 μl reaction buffer (10× Taq extender buffer; Perkin-Elmer Biosystems);
The reaction volume was made up to 80 μl with water treated with DEPC. All work stages took place on ice. The reaction vessels are shaken briefly and incubated in a thermocycler model 2400 (Perkin-Elmer Biosystems) in accordance with the following amplification programme:

Reverse Transcription

The reverse transcription was carried out at 50° C. for 60 min, the denaturing and isolation of the nucleic acid strings required temperatures of 94° C. for 30 s. At 55° C., the primers accumulated on sense or antisense strings (annealing); DNA synthesis (elongation) occurred at 72° C. (10 min).

PCR Cycles

35 cycles, 30 s at 94° C., 2 min at 55° C. and 7 min at 72° C.

Termination: 20 min at 72° C., storage at 4° C.

After amplification, 10 μl of the reaction mix with 5 μl BSE buffer was spread on a 1% agarose gel and electrophoretically separated in TAE buffer at 120 volts. The gel was then dyed for 15 min in an ethidium bromide bath and analysed under ultraviolet light (UV) at a wavelength of 1=301 nm. The DNA strips were compared qualitatively with the length standard (1 kb DNA Ladder, New England BioLabs) and the quantities estimated.

In the same way as described, RNA was also isolated from preserved mucous membrane and secretion samples and from umbilical cord tissue samples (TFS tissue sampling system) and the BVD virus detected.

2. Processing Using Own Processes

Detecting BVDV in Typifix Tissue Samples

Detecting BVDV in animal tissue was carried out using RT PCR. Firstly, the RNA was purified out of the samples. Magnetic particles with a silica surface were used for this purpose. A specific fragment of viral RNA was then transcribed into cDNA in two separate enzymatic reactions by reverse transcriptase and exponentially multiplied by polymerase chain reaction. The analysis was carried out using an ELISA-type process in which the specific amplificates were detected directly in the PCR vessels by a biotin streptavidin detection reaction with an enzyme conjugate and a subsequent enzyme reaction. In the enzyme reaction, a substrate was converted into a coloured product. The optical density was then measured in a conventional ELISA reader.

Purifying Total RNA from Typifix Samples

The tissue samples were preserved in RNAlater. The samples were transferred into 1.5 ml reaction vessels with screw lids to be broken down. In the reaction vessels, there were glass spheres to break down the tissue.

100 μl of a ready-to-use lysis buffer A was pipetted into each of the samples. The vessels were shut tight and clamped into the fixing device of a vibration grinding mill (RETSCH). The tissue was then broken down with the glass spheres for 4 min at a frequency of 30 sec-i in the vibration grinding mill. The reaction vessels were then centrifuged briefly at 5,000×g. Then 900 μl of lysis buffer B was added and the lysates mixed by inverting the reaction vessels. The excess above the glass spheres, which settle quickly, was removed with a pipette and transferred into prepared reaction vessels containing magnetic particles. The magnetic particles were re-suspended in the lysis mix by vortexing thoroughly. To bind the RNA to the magnetic particles, the reaction vessels were then incubated for 2 min at room temperature. To immobilise the magnetic particles, the reaction vessels were then placed in a magnetic stand. The excess was removed and discarded. The reaction vessels were then removed from the magnet and 500 μl of ice-cold 70% ethanol was pipetted into each vessel. The magnetic particles were washed by vortexing and then immobilised once again in the magnetic stand. The washing solution containing ethanol was completely removed and discarded. To remove any residues of ethanol, the reaction vessels were taken out of the magnetic stand and left to stand open for 5 min at room temperature. The magnetic particles were then re-suspended in 60 μl RNAse-free purified water (by vortexing briefly) and the RNA separated in a thermo-agitator (agitation frequency of 400 rpm) by incubation for 2 min at 65° C. The reaction vessels were then cooled on ice for 2 min. The magnetic particles were subsequently immobilised once again in the magnetic stand, and the excess containing RNA was removed and transferred into clean reaction vessels. During the triggering of the cDNA synthesis reactions, the cleaned RNA was cooled on ice.

cDNA Synthesis by Reverse Transcription

The cDNA synthesis was carried out by reverse transcription with MMLV reverse transcriptase (RNAse H minus). A BVDV-specific oligonucleotide (BVDV 326) was used as the primer.

To hybridise the virus RNA with the primer, 5 μl each of the cleaned total RNA preparations from the tissue samples was mixed with 2 μl primer (10 μmol/μl) and 5 μl purified water in thin-walled 0.2 ml reaction vessels. The mixes were then incubated in a PCR cycler block for 10 min at 70° C. and then cooled rapidly to 10° C. During the incubation, an enzyme buffer mixture was prepared with the following composition:
4 μl RT buffer 5× concentrated
1 μl dNTP mix (10 mM of each of the 4 deoxynucleoside triphosphates)
0.4 μl enzyme mixture (consisting of 25 u/μl reverse transcriptase and 10 u/μl RNAsin)
2.6 μl purified water.

The mixture is prepared a number of times depending on the number of samples.

8 μl of the prepared mixture was pipetted into the cooled RNA primer mixtures (see above) and mixed briefly by vortexing. The cDNA synthesis then took place for 30 mins. at 37° C. in a PCR cycler block. After synthesis, the reverse transcriptase was inactivated by heat (5 min at 75° C.). The cDNA was then cooled directly on ice and put in a PCR. Alternatively, the cDNA samples can be frozen at −20° C. and further processed at a later date.

Amplification of a Specific Fragment of the BVDV Genome by PCR and Analysis

From the cDNA preparations, a fragment of the BVDV genome was multiplied with the said primers in a polymerase chain reaction (PCR). In a 96-well plate, composed of 12 strips each with 8 reaction cavities, a so-called PCR premix was pipetted. The premix consisted of 2 components—the PCR buffer mix and the PCR enzyme mix. In the mixes, all components for the PCR are contained, kit-like, in a form geared to the detection process. On the plastic surface of the reaction cavities, specific oligonucleotides were bound covalently for detection of the BVD virus. They were used as “traps” for BVDV-specific amplificates in the detection.

The premix was prepared a number of times depending on the number of samples to be analysed, including the associated controls. In a 2-ml reaction vessel, 19.91 μl PCR buffer mix and 0.09 μl PCR enzyme mix were mixed per sample. Then, 20 μl of the premix was distributed into each of the reaction cavities on the strips of 8 using a dispenser pipette. Then, 2 μl of the cDNA samples (see above) was pipetted into each of the cavities.

The plate was then sealed with Nunc sealing tape and placed in a 96-cavity block of a PCR cycler (PE Biosystems, PCR System 9700) with a heated lid. A programme was started with the following parameters:

| | |
|---|---|
| 94° C.-5 min | |
| 94° C.-30 sec | |
| 50° C.-30 sec | |
| 72° C.-30 sec | 35 cycles of steps 2-4 |
| 72° C.-2 min | |
| 94° C.-3 min | |
| 56° C.-5 min. | |

After the end of the programme, the reaction cavities in the strips were washed 5 times in an ELISA washer with washing buffer (component of the kit). The residues of the washing buffer were removed by “knocking out” on filter paper. Using a multi-channel pipette, 50 μl of a ready-to-use streptavidin peroxidase conjugate was then pipetted into each cavity and this was incubated for 15 min at room temperature. Non-bound conjugate was then removed by washing 5 times with washing buffer. After “knocking out” on filter paper once again, 25 μl of a substrate solution (TMB) was then pipetted into each cavity and incubated for 15 min in the dark at room temperature. The substrate (TMB) was converted into a blue-stained product with peroxidase. The optical density of the blue-stained solution in the cavities was measured photometrically in an ELISA reader at a wavelength of 650 nm.

Using the results of measurements for the negative controls, a threshold value was calculated statistically. If the measured values for the samples were above the threshold, BVDV had been present. The efficiency of the RNA purification, cDNA synthesis and PCR was indicated by corresponding positive controls.

Analysis

With all samples from persistently infected BVD calves, the BVD virus could be detected. No difference was to be found between preservation with trizol and the less toxic and less highly volatile RNAlater.

All currently known BVD genotypes were detectable. Detection was possible not only in tissue samples tested within a few days after they were taken, but also possible in samples obtained with the RNAlater TFS sampling system and which had been stored at room temperature for several weeks and months.

In all cases, the bands detected in the analysis of the tissue sample were just as meaningful as the bands of the positive controls also tested, coming from leucocyte pellets from calves' blood samples. The sensitivity and specificity of the BVD diagnoses from the tissue samples allows persistently infected BVD calves to be detected and thereby enables these animals to be identified early on and reliably—before the diagnostic gap—and, by removing them from the herds, enables the infection to be eradicated from these herds—and in the event of population-wide implementation—from an entire country.

### EXAMPLE 2

Extraction of Vital Cells using Typi-Fix Sample Collecting Containers.

To extract vital cells, it is necessary to place the tissue sample obtained by the stamping process in an appropriate medium. To do this, both the collection container and the hollow point of tissue sampling systems were filled with cell culture medium and the containers sealed tight. TL Hepes with BSA containing penicillin G (0.0325 g/l) to which gentamycin (0.025 g/l) was mixed, was used as the medium.

Collecting containers filled with medium were used to collect samples from cattle and sheep and to transport them to the laboratory. There, the samples were removed from the collecting containers and a cell culture was prepared using a known process. The fibroblasts growing in the cell culture were used to perform a cloning by nucleus transfer. After the transfer of the cells cloned from the preserved samples, pregnancies and offspring arose, whereby the process disclosed in EP 98 907 950.4 which is incorporated herein by reference, was used.

1. A device for extracting biological samples, comprising a sample collecting container and a sample extraction means, wherein an agent to protect against RNA-degrading enzymes is provided in the sample collecting container and the sample extraction means.

2. The device according to claim 1, wherein the agent to protect against RNA-degrading enzymes is provided in the sample collecting container in a fitting which is arranged on the base of the sample collecting container.

3. The device according to claim 2, wherein the fitting is a membrane through which the agent to protect against RNA-degrading enzymes is held at the base of the sample collecting container, or a porous carrier.

4. The device according to claim 3, wherein the porous carrier is a sponge.

5. The device according to claim 1, wherein the sample extraction means is coated with the agent to protect against RNA-degrading enzymes or has a cavity in which the agent to protect against RNA-degrading enzymes is provided and which is separated from the environment by a membrane.

6. The device according to claim 1, wherein the agent to protect against RNA-degrading enzymes is DEPC, guanidinium hydrochloride, trizol, molecular sieve or RNAlater.

7. A process for the detection of an attack on an individual by an RNA virus, comprising
obtaining a biological sample from the individual to be tested by means of the device of claim 1, and
testing the biological sample for the presence of the RNA virus.

8. The process according to claim 7, wherein the RNA virus is BVD, PRRS or TGE.

9. (Canceled)

10. (Canceled)

11. A sampling set, comprising at least one device according to claim 1 or a sample collecting container therefrom and identification components.
